(19) United States
(12) Patent Application Publication
(10) Pub. No.: US 2007/0173944 A1
(43) Pub. Date: Jul. 26, 2007

(54) Endoprosthesis with intermediate part

(75) Inventors: Arnold Keller, Kayhude (DE); Hakon Kofoed, Charlottenlund (DK)

Correspondence Address: MORRISON & FOERSTER LLP, 1650 TYSONS BOULEVARD, SUITE 400, MCLEAN, VA, 22102, US

(73) Assignee: Waldemar Link GmbH & Co. KG, Hamburg (DE)

(21) Appl. No.: 11/637,238

(22) Filed: Dec. 12, 2006

Related U.S. Application Data
(60) Provisional application No. 60/749,103, filed on Dec. 12, 2005.
(60) Provisional application No. 60/755,079, filed on Jan. 3, 2006.

(51) Int. Cl. A61F 2/30

(52) U.S. Cl. 623/18.11; 623/20.33

(57) ABSTRACT

The invention relates to an endoprosthesis for replacement of a joint, 2 slide surfaces having different contours and correspondingly determining movement planes for in each case 1 bearing being formed by an intermediate part 2. Provision is made according to the invention for a clamping bracket to enclose the slide surfaces of the intermediate part and to be arranged thereon such that it is arranged free from the movement planes defined by the slide surfaces having different contours. It is thus also possible to strengthen endopreostheses which have complex biomechanics having a plurality of degrees of freedom.

### REFERENCE TO RELATED APPLICATIONS

This application claims the priority of U.S. Provisional Application Ser. Nos. 60/749,103, filed Dec. 12, 2005, and 60/755,079, filed Jan. 3, 2006, the contents of both of which prior applications are incorporated herein by reference.

### FIELD OF THE INVENTION

The invention relates to an endoprosthesis for replacement of a joint, comprising a component which is to be connected to a lower bone and which has a top slide surface, a component which is to be connected to an upper bone and which has a bottom slide surface, and an intermediate part which, on its bottom and top, has in each case a matching slide surface which, together with the slide surfaces of the aforementioned components, in each case forms a bearing.

### BACKGROUND OF THE INVENTION

Endoprostheses of this kind are used, for example, for replacement of the ankle joint (FR-A 2 676 917, WO-A-03/075802, WO-A-2005/030098). In these, the components and the intermediate part cooperate via slide surfaces which permit flexion and extension in a sagittal plane. The sagittal plane is in this case a plane which is defined by AP direction and the vertical axis. The tibial component and the intermediate part form interacting slide surfaces which permit a rotation about the vertical axis. They can have a plane configuration in order to permit compensating movements in the AP direction and LM (lateral-medial) direction. So that the joint has degrees of freedom with respect to rotary, pivoting and/or shearing movements, which degrees of freedom correspond to the natural model, the slide surfaces correspondingly have different contours, for example a flat slide surface is combined with a slide surface which is curved in the manner of a cylindrical sleeve. Stabilization is afforded by the natural ligament apparatus.

The full load of the endoprosthesis rests on the intermediate part. The latter is therefore subject to substantial loading. In practice it has been found that substantial loads can result in a “warping” of the intermediate part normally made of polyethylene. There is therefore a danger that, under increased loads, as may occur for example as a result of movement dynamics (in particular when climbing stairs or jumping), the intermediate part will become overloaded and thus suffer uncontrolled deformation. This can lead to excessive wear, cold flow, or even malfunction of the prosthesis as a result of material failure. This is in particular the case if the intermediate part has a thin design, as is usually the case as a consequence of the different contours of the slide surfaces.

### SUMMARY OF THE INVENTION

Starting out from the cited prior art, the object of the invention is to improve an endoprosthesis of the type mentioned in the introduction in such a way that it can more reliably withstand high loads while maintaining multiple degrees of freedom.

The solution according to the invention lies in a prosthesis including a first bone component configured for connection to a lower bone and having a bottom slide surface, a second bone component configured for connection to an upper bone and having a top slide surface, an intermediate part having slide surfaces on its bottom and top which, together with the slide surfaces of the first and second bone components define a movement plane for a bearing, the slide surfaces of the intermediate part having different contours, and a clamping bracket enclosing side surfaces of the intermediate part and arranged on the intermediate part free from the movement planes defined by the slide surfaces of the intermediate part. Advantageous developments are explained in the detailed description below.

Accordingly, in an endoprosthesis for replacement of a joint, comprising a component which is to be connected to a lower bone and which has a top slide surface, a component which is to be connected to an upper bone and which has a bottom slide surface, and an intermediate part which, on its bottom and top, has in each case a slide surface which, together with the slide surfaces of the aforementioned components, in each case define a movement plane for a bearing, the slide surfaces of the intermediate part having different contours, the invention proposes that a clamping bracket encloses the side surfaces of the intermediate part and is arranged on the intermediate part free from the movement planes defined by the slide surfaces having different contours. The term movement plane is to be interpreted in a broad sense and also includes curved contours.

With the clamping bracket acting as a belt of greater tensile strength compared to the polyethylene, the resulting overall modulus of elasticity of the intermediate part is increased. The clamping bracket is for this purpose expediently made from a material such that it has an at least fifty times, preferably at least two hundred times, greater modulus of elasticity than the polyethylene. With the clamping bracket acting as a belt with tensile strength, elastic or plastic deformation of the intermediate part under loading is counteracted. The intermediate part can thus also withstand greater loads without deforming. Thus, the intermediate part can be strengthened with the clamping bracket according to the invention. By virtue of the inventive design of the clamping bracket, the movement surfaces, as are defined by the slide surfaces of the upper and lower bearing, remain free. Thus, despite the strengthening by the clamping bracket, the mobility of the endoprosthesis is maintained in all functions of the joint. This applies not only to the normal range of movement of the endoprosthesis, but also to movements going beyond these, such as may occur for example upon twisting of the foot.

It has of course been made known for polyethylene plateaus, functioning as part of knee-joint endoprostheses, to be strengthened by providing a metal plate on the underside of the plateau resting on the tibia. In this way, the polyethylene plateau has been strengthened from its rear face in such a way that it yielded less under flexural stress. However, this strengthening measure known from EP-A-0 829 243 can be used only in prostheses which have a slide surface on just one side. In an endoprosthesis of the type in question here, the intermediate part has slide surfaces on its top and also on its bottom, thus ruling out the use of such a strengthening plate. The same applies to a strengthening ring as disclosed in US-A-5 766 256. This ring too is arranged on the bottom face, which does not serve as a joint surface.

The clamping bracket is preferably designed with two zones, namely with a belt zone extending in the circumferential direction and a spread protection zone which adjoins the belt zone. In this connection, the spread protection zone does not have to be provided all round the circumference, and instead it generally suffices for it to be provided on two opposite sides. The spread protection zone additionally counteracts a divergence of the outer portions of the intermediate part under high flexural stress.

The top and bottom edge of the clamping bracket are preferably adapted to the contour of the respectively adjacent slide surface. Adapted is here understood as meaning that, seen in a side view, the edge of the clamping bracket is at a constant distance from the edge of the adjacent slide surface. If the one slide surface is for example a plane, then its edge is a straight line and the corresponding edge of the clamping bracket is likewise a straight line; if the other slide surface is correspondingly curved, its edge is in the shape of an arc of a circle and the corresponding edge of the clamping bracket is likewise an arc of a circle with an edge in the shape of an arc of a circle. The top and bottom edges of the clamping bracket being adapted to the respective contour makes it possible to achieve a strengthening of the particularly loaded intermediate part, even in the case of endoprostheses having complex joint function, such as those having slide surfaces with different contours, and still to ensure that the element effecting the strengthening, the clamping bracket, remains free from the complex movement planes defined by the slide surfaces with different contours.

The clamping bracket at its bottom edge expediently has, at least on two sides, a bevel which is configured such that it merges smoothly into the intermediate part. On its inner side, the clamping bracket also preferably has a bead-like projection which engages in a corresponding recess on the intermediate part. The clamping bracket is thus secured against an undesired displacement from its intended position. However, other securing techniques can also be provided, for example adhesive bonding or binding, generated in particular by shrinking the clamping ring onto the intermediate part. A form-fit connection can also be provided, for example pinning or screwing.

In order to give the clamping bracket a defined position on the intermediate part, it has, on its top or bottom, a flange on which the clamping bracket bears. This makes fitting of the clamping bracket easier, since the intended position is clearly defined. This also has the effect that the slide surface of the intermediate part does not have to be made smaller because of the clamping bracket. In this way, the surface load is not any greater than in the conventional design of the intermediate part without clamping bracket.

The clamping bracket is expediently dimensioned such that its top edge and its bottom edge are at a distance of at least 1 mm, preferably between 1.5 and 2.5 mm, from the edge of the respective slide surface. This ensures that, even in the event of a high load leading to compression of the intermediate part, or in the event of wear of the intermediate part, it is possible to avoid undesired contact between the clamping bracket and the slide surfaces of the components of the prosthesis.

According to a particularly preferred embodiment, which possibly merits independent protection also for endoprostheses having slide surfaces with the same contour, the clamping bracket has a convex projection on at least one outer face. The effect of the projection is that, in the event of a rotation, as also in a linear movement, of the joint and thus also of the intermediate part and clamping bracket, undesired tissue material growing laterally alongside the endoprosthesis can be forced back. It is in this way possible to counteract or even prevent infiltration of this tissue material, so-called fibrosis. The danger of the joint with the endoprosthesis according to the invention having its mobility restricted by excessive fibrosis can thus be averted. Pain which can normally occur in the event of such fibrosis, on account of the tissue material growing in the area of movement of the intermediate part, is avoided by virtue of the development according to the invention. By virtue of the configuration according to the invention, an otherwise unavoidable surgical removal of this tissue material is unnecessary.

A particular advantage of this development is that, with the clamping bracket preferably made of metal, a contact with the bone or the tissue material can in principle take place, whereas, in the intermediate parts customarily made exclusively of polyethylene material, a contact with the bone or the tissue material was not desirable, because this leads to undesired polyethylene abrasion. The configuration according to the invention thus makes use of the clamping bracket in two ways, namely its structure for forming the convex projection for forcing back the fibrosis, and its material which for the first time permits contact with the fibrotic tissue material.

The convexity of the projection need only be one-dimensional, such that an essentially cylindrical shape is obtained; however, it can preferably also be two-dimensional, such that an essentially spherical configuration is obtained, in which case the curvature in the plane of the clamping bracket and perpendicular thereto can be different.

The convex projection expediently extends across the entire length of the respective outer face. Although the desired effect can in principle also be achieved with a projection extending over only part of the length of an outer face, greater and therefore more favorable radii of curvature for the projection arise in a design across the entire length. An arrangement of the convex projection on a medial longitudinal face of the clamping bracket is particularly expedient. In the case of an implantation of the endoprosthesis according to the invention on the ankle joint for example, the medial malleolus is situated in this area. It is in this very area that undesired fibrosis may occur, the damaging results of which can be prevented by virtue of the development according to the invention. The arrangement extending across the entire length also has the advantage that the desired effect of the forcing back can be achieved also in a non-rotational movement, for example a linear forward and rearward movement of the intermediate part.

The contour of the convex projection is expediently chosen such that it has the shape of an arc of a circle in plan view. Such a contour is favorable in production and gives a uniform curvature of the projection without pronounced changes to the curve profile. It is not necessary here for the center of the circle arising from the arc to lie centrally in the clamping bracket. It is expediently offset in the direction of the opposite lateral face. This results in an eccentricity, on the basis of which a stronger forcing back of the fibrotic tissue material is achieved with greater rotatory deflections of the intermediate part.

The outer face of the convex projection is preferably smooth. It can preferably be polished. This gives a form that promotes sliding, in particular under the influence of tissue fluid. The danger of tissue material being torn off is thus effectively counteracted.

The convex projection can expediently also be provided on the adjacent outer faces. In the case of a rectangular design, this means that such a convex projection is formed on the anterior face, the posterior face and the medial face of the clamping bracket.

### BRIEF DESCRIPTION OF THE DRAWINGS

The invention is explained below with reference to the attached drawing which depicts an advantageous illustrative embodiment of the invention and in which:

FIG. 1 shows a sagittal section through an ankle joint fitted with the prosthesis according to the invention;

FIG. 2 shows the prosthesis according to FIG. 1 in a perspective view and opened out;

FIGS. 3a), b) show a front view and a side view, respectively, of a clamping bracket of the prosthesis;

FIG. 4 shows a partial cross-sectional view of the clamping bracket with an intermediate part of the prosthesis;

FIG. 5 shows a front view of a lower area of the shin bone with a part of a variant of the endoprosthesis according to FIGS. 1 to 4;

FIG. 6 shows a bottom view of the variant according to FIG. 5, and

FIGS. 7a), b) show a front view and side view, respectively, of the clamping bracket shown in FIG. 6.

### DETAILED DESCRIPTION OF THE INVENTION

The depicted illustrative embodiment of the prosthesis according to the invention is an ankle joint prosthesis. It will be noted that the invention can also be applied to other types of endoprostheses, for example intervertebral endoprostheses. The important point is that the endoprosthesis has two bearings whose planes of movement are defined by the slide surfaces having different contours.

The endoprosthesis according to the depicted illustrative embodiment basically comprises three components. The first component is a shin bone component 1 designed to be arranged on the lower end of a tibia 91. It has a plate-shaped part 10 whose bottom forms a plane slide surface 11. On the top of the plate-shaped part 10 there is an anchoring body 12 which is provided with projections and which serves to secure the shin bone component 1 in corresponding resection depressions in the tibia 91.

The prosthesis further comprises an ankle bone component 4. It has a saddle-like configuration and has a convexly curved slide surface 44 on its top. It can be configured in the manner of a jacket of a cylinder, as shown here. However, it can equally well have a cone-shaped design. A guide rib 46, which lies in the AP direction, is arranged on it. This serves for guiding purposes in a flexion and extension movement of the ankle joint.

An intermediate part 2 is arranged between the shin bone component 1 and the ankle bone component 4. On its top, it has a plane slide surface 21 which is configured to match the slide surface 11 of the shin bone component 1. On its bottom, the intermediate part 2 has a slide surface 24 which is congruent to the slide surface 44 of the ankle bone component 4. It additionally has a groove 26 which is designed to receive the rib 46 in a longitudinally displaceable manner. In this way, the intermediate part 2 is guided laterally in relation to the ankle bone component 4. Only flexion and extension movements are thus permitted. By contrast, the plane slide surfaces 11, 21 permit any desired movement in a horizontal plane, that is to say both longitudinal and transverse movements and also, in particular, a rotation about the vertical axis.

The shin bone component 1 and the ankle bone component 4 are expediently made of metal, for example a cobalt-chromium alloy provided on its respective outer face with a coating that promotes bone growth (for example calcium phosphate). The intermediate part 2, by contrast, is preferably made of a plastic material that promotes sliding, in particular polyethylene.

However, this is not intended to rule out the possibility of also using other materials with sufficient strength and slidability.

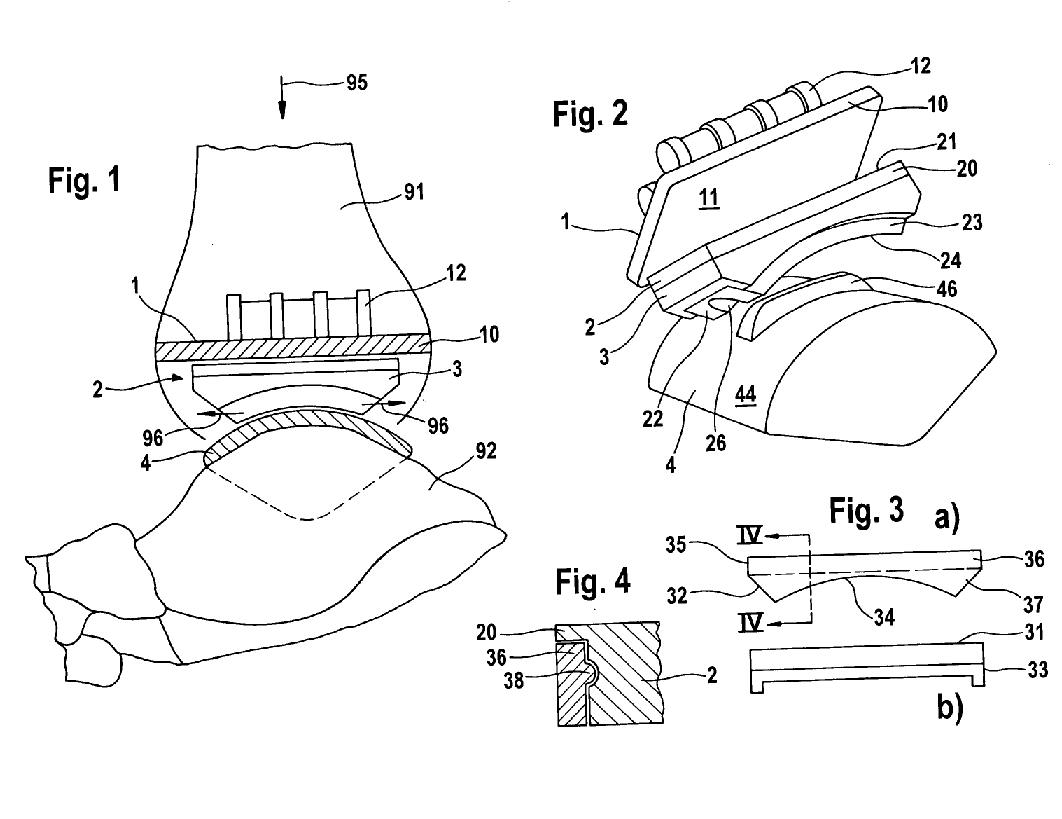

In the implanted state, the joint, and in particular the intermediate part 2, is subjected to a high axial load (symbolized by an arrow 95) along the vertical axis. On account of the resulting compression, a horizontally outwardly directed divergent force arises in the polyethylene material of the intermediate part 2 (as symbolized in FIG. 1 by the arrows 96). This divergent force is further intensified by the convex configuration of the slide surface 44 of the ankle bone component 4. High loads may therefore result in an undesired deformation of the intermediate part 2.

To counteract this, a clamping bracket 3 according to the invention is provided. It is made of a cobalt-chromium alloy with a modulus of elasticity which is approximately four hundred times as high as that of the polyethylene material of the intermediate part 2. It is also possible to use titanium, which has an approximately two hundred times higher modulus of elasticity. The clamping bracket 3 is made from a flat strip material. It has a thickness of 1 mm, for example. In horizontal section, the clamping bracket 3 has a contour corresponding to the outer contour of the intermediate part 2. In the illustrative embodiment shown, this is a quadratic contour. However, another contour could equally well be provided, for example a round one in the case of a design as an intervertebral prosthesis. Its dimensions are chosen such that it tightly encloses the intermediate part 2.

In its upper part, the clamping bracket 3 has a circumferential belt zone 36. This counteracts a deformation of the intermediate part 2 in all lateral directions (in longitudinal direction and in transverse direction) under loading. In addition, the bottom of the belt zone 36 is adjoined by a spread protection zone 37. The spread protection zone 37, to which the bevel 32 also belongs, additionally stabilizes the outer areas of the concave slide surface 24 and thus counteracts in a particularly effective manner the divergent force component 96 resulting from the convex configuration of the slide surface 44. The intermediate part 2 is thus strengthened by virtue of the clamping bracket according to the invention. Thus, even in the event of a high load, undesired bending deformation is counteracted.

The clamping bracket 3 is straight at its top edge 31. This results in a constant distance from the edge of the top slide surface 21. On its bottom edge 34, the clamping bracket has an arcuate configuration on its longitudinal sides 33 (which are oriented parallel to the rib 46). It is configured such that in this area there is a constant distance of the bottom edge 34 of the clamping bracket 3 from the edge of the lower slide surface 24. On its transverse sides 35, the clamping bracket 3 has a straight bottom edge. This once again results in a constant distance from the corresponding edge of the slide surface 24. In the area of the transverse sides, the bottom edge of the clamping bracket is extended downward like an apron and also has a bevel 32. The latter is configured such that it forms a continuous plane with the corresponding side surface 22 of the intermediate part 2. The bevel provides additional strengthening specifically in an area which is particularly loaded by the divergent forces (see arrow 96), and specifically in a way that does not involve undesired restriction of mobility.

The intermediate part 2 has a flange 20 in the area of its upper slide surface 21. The clamping bracket 3 is pushed flush onto the underside of the flange 20 in such a way that a smooth transition is formed on the outside between the flange 20 and the outer face of the clamping bracket 3.

By virtue of the inventive configuration of the clamping bracket 3, the upper and lower slide surfaces 21, 24 remain free, such that their bearing function is not adversely affected.

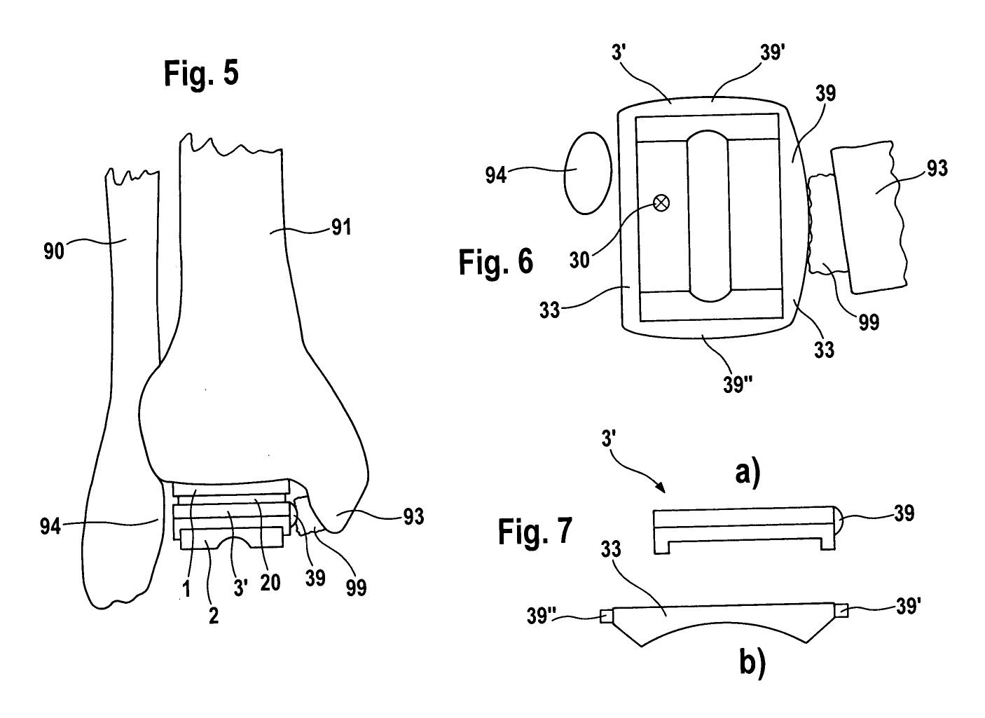

As a further illustrative embodiment, FIGS. 5 to 7 show a variant of the ankle-joint endoprosthesis according to FIGS. 1 to 4. FIG. 5 shows the endoprosthesis at its intended implantation site on the distal end of the tibia 91. For the sake of clarity, the only parts of the endoprosthesis that are shown here are the shin bone component 1, the intermediate part 2 and a varied clamping bracket 3′. The fibula 90 runs parallel to the tibia 91. At its distal end, the tibia 91 forms a plateau on which the shin bone component 1 of the endoprosthesis according to the invention is arranged. This plateau is limited in the medial direction by a continuation of the tibia 91, the so-called medial malleolus 93, and in the lateral direction by a corresponding continuation of the fibula 90, namely the lateral malleolus 94. They enclose the plateau of the tibia 91 and therefore the shin bone component 1 of the endoprosthesis like a fork. This can be seen clearly in FIG. 6.

It has been shown that, some time after implantation, a formation of tissue material (fibrosis) 99 often occurs in the area between the medial malleolus 93 and the intermediate part 2 or the clamping bracket 3′ arranged around the latter. This can cause pain which not only could be very unpleasant for the patient but in quite a few cases could also necessitate a surgical intervention to remove the tissue material 99. To avoid or reduce the fibrotic tissue material 99, a projection 39 is formed at least on a longitudinal face 33 of the clamping bracket 3′, expediently on the medial face. The projection extends outward relative to a contour which is congruent with the intermediate part 2. A projection of this type which projects over the congruent contour may also be provided on endoprostheses which have slide surfaces having the same contours. The projection preferably has an arc-shaped outer contour, the arc extending across the entire length. The projection 39 is expediently curved in two dimensions, that is to say it has a spherical surface shape (see FIG. 7a). The radii of curvature are of different sizes, a weak curvature in the horizontal plane (as is shown in FIG. 6) and a stronger curvature in a frontal plane (as is shown in FIG. 7a). To obtain the largest possible radius of curvature in the horizontal plane, the midpoint of the circle defined by the radius of curvature preferably does not lie centrally in the clamping bracket 3′ but is instead eccentrically offset in the lateral direction and preferably also in the frontal direction. The outer face of the projection 39 is smooth.

The illustrative embodiment shown represents one option, specifically one in which the front face and the rear face of the clamping bracket 3′ are also each provided with a projection 39′ and 39″, respectively. They are expediently configured corresponding to the projection 39, but can also deviate from this in shape (e.g. cylindrical instead of spherical, as is shown in FIG. 7b). A transition of equal curvature between the projections 39, 39′, 39″ is not necessary, but the geometries are expediently chosen such that the transition is stepless. The lateral longitudinal face of the clamping bracket 3′ expediently has no projection. This serves to ensure free movement of the clamping bracket. This also has the advantage of providing an unambiguous orientation of the clamping bracket 3′, as a result of which the danger of its being fitted in an incorrect position is reduced. Like the clamping bracket 3 of the illustrative embodiment shown in FIGS. 1 to 4, the clamping bracket 3′ is preferably made of a metal material, in particular titanium or a cobalt-chromium alloy. It can thus come into contact with the fibrotic tissue material 99 without there being any risk of its adversely affecting the surrounding tissue. Upon movement of the endoprosthesis, in particular upon rotation, but also upon movement in the longitudinal direction toward the front or rear, the projection 39 ensures that the fibrotic tissue material 39 is forced back. This therefore effectively counteracts growth of the fibrotic tissue material 99 into the area of the endoprosthesis.

The projection 39 is normally designed in one piece with the clamping bracket 3′. However, this should not rule out the possibility of choosing a multi-part construction in which the projection 39 is designed as a separate part and is secured on the clamping bracket 3′ by suitable securing means. The latter affords the advantage that, for the projection 39, it is possible to choose a material which especially promotes sliding and is especially suitable for contact with the fibrotic tissue material 99, without concerning oneself about its mechanical load-bearing capacity as strengthening element, as is important for the choice of the material for the clamping bracket 3.

Finally, it will be noted that the configuration according to the invention of a clamping bracket with a projection 39 is not limited to ankle-joint endoprostheses.

1. An endoprosthesis for replacement of a joint, comprising:
a first bone component configured for connection to a lower bone and having a bottom slide surface,
a second bone component configured for connection to an upper bone and having a top slide surface,
an intermediate part having slide surfaces on its bottom and top which, together with the slide surfaces of the first and second bone components, define a movement plane for a bearing, the slide surfaces of the intermediate part having different contours, and
a clamping bracket enclosing side surfaces of the intermediate part and arranged on the intermediate part free from the movement planes defined by the slide surfaces of the intermediate part.

2. The endoprosthesis as claimed in claim 1, wherein top and bottom edges of the clamping bracket are adapted to the contour of the respective adjacent slide surfaces of the intermediate part.

3. The endoprosthesis as claimed in claim 1 or 2, wherein the clamping bracket is configured to have a belt zone extending in a circumferential direction and a spread protection zone which adjoins the belt zone.

4. The endoprosthesis as claimed in claim 1 or 2, wherein the intermediate part comprises a flange on which the clamping bracket is configured to bear.

5. The endoprosthesis as claimed in claim 1 or 2, wherein the clamping bracket at its bottom edge has, at least on two sides, a bevel which is configured such that it merges smoothly into the intermediate part.

6. The endoprosthesis as claimed in claim 1 or 2, wherein the top and bottom edges of the clamping bracket are spaced apart by a distance of at least 1 mm.

7. The endoprosthesis as claimed in claim 1 or 2, wherein the clamping bracket has, on its inner side, a bead-like projection which engages in a corresponding recess on the intermediate part.

8. The endoprosthesis as claimed in claim 1 or 2, wherein the clamping bracket has a modulus of elasticity at least fifty times greater than a modulus of elasticity of the intermediate part.

9. The endoprosthesis as claimed in claim 1 or 2, wherein the clamping bracket has a convex projection formed on at least one outer face.

10. The endoprosthesis as claimed in claim 9, wherein the convex projection extends across the entire length of the respective outer face.

11. The endoprosthesis as claimed in claim 9, wherein the projection is formed on a medial longitudinal face of the intermediate part.

12. The endoprosthesis as claimed in claim 9, wherein the convex projection has an arc-shaped contour in plan view.

13. The endoprosthesis as claimed in claim 12, wherein the arc-shaped contour follows an arc of a circle whose center is offset toward an opposite side of the clamping bracket.

14. The endoprosthesis as claimed in claim 9, wherein additional convex projections are formed on an anterior face and a posterior face of the clamping bracket.

15. The endoprosthesis as claimed in claim 6, wherein the top and bottom edges of the clamping are spaced apart by a distance of 1.5 to 2.5 mm.

16. The endoprosthesis as claimed in claim 8, wherein the clamping bracket has a modulus of elasticity at least two hundred times greater than a modulus of elasticity of the intermediate part.
